(19) United States
(12) Patent Application Publication
(10) Pub. No.: US 2020/0373004 A1
(43) Pub. Date: Nov. 26, 2020

(54) GEOGRAPHICALLY VERIFIED PATIENT LOGS

(71) Applicant: Doc Technologies Inc., Chicago, IL (US)

(72) Inventors: Jamil Elkoussa, Orland Park, IL (US); Sameer Suhail, Chicago, IL (US); Abdulwahab Omira, Darien, IL (US); Mohannad Sandid, Darien, IL (US)

(21) Appl. No.: 16/417,185

(22) Filed: May 20, 2019

(51) Int. Cl. G16H 40/20; G06F 16/29

(52) U.S. Cl. CPC G16H 40/20; G16H 10/60; G06F 16/29

(57) ABSTRACT

Systems, methods, and computer-executable instructions for receiving geographically verified patient logs that includes receiving, from a medical application, a check-in indication associated with a student. The check-in indication includes a hospital identifier that identifies a hospital. A geographic location of the hospital is determined based on the hospital identifier. The geographic location of the hospital is sent to the medical application in response to the check-in indication. A geographically verified patient log is received from the medical application. The patient log is geographically verified based on the geographic location of the hospital. The geographically verified patient log is stored.

### BACKGROUND

Medical clerkships include clinical rotations that give medical students the ability to apply classroom knowledge to real-world medical situations. As part of a clerkship, a student works in a hospital where the student directly interacts and engages patients under the supervision of physicians. Typically, a student will rotate through different specialties and treat patients with various conditions. As part of treating patients, students complete patient logs detailing a student's interaction with a patient. The patient logs are used to track a student's progress and are used by the administration of the clerkship.

### BRIEF DESCRIPTION OF DRAWINGS

The features of the present disclosure will become more fully apparent from the following description and claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 is a block diagram of system for geographically verifying patient logs in accordance with respective examples.

FIG. 2 is a flow diagram of a process for receiving geographically verified patient logs in accordance with respective examples.

FIG. 3 is a flow diagram of a process for providing geographically verified patient logs in accordance with respective examples.

FIG. 4 is an example computing device that can be used in conjunction with the technologies described herein.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and made part of this disclosure.

### DETAILED DESCRIPTION

Patient logs are entered by medical students taking part in a medical clerkship. Common practice is for students to enter patient logs on a regular, e.g., weekly, bi-daily, daily, etc., basis. Hard copies of patient log forms may be used to collect patient information. As portable wireless devices have become common, applications on a portable wireless device may also be used to collect patient logs. Technology has also allowed students to take classes remotely rather than in-person. One downside to such technological advances is students creating patient logs when they are no longer at their hospital and long after the logged patient interaction. Such delayed input may result in incomplete or less informed patient logs. In addition, a reviewing physician or administrator will not know that the patient log was delayed. Verifying that a patient log was done within the vicinity of the hospital is one way to document the validation of a patient log.

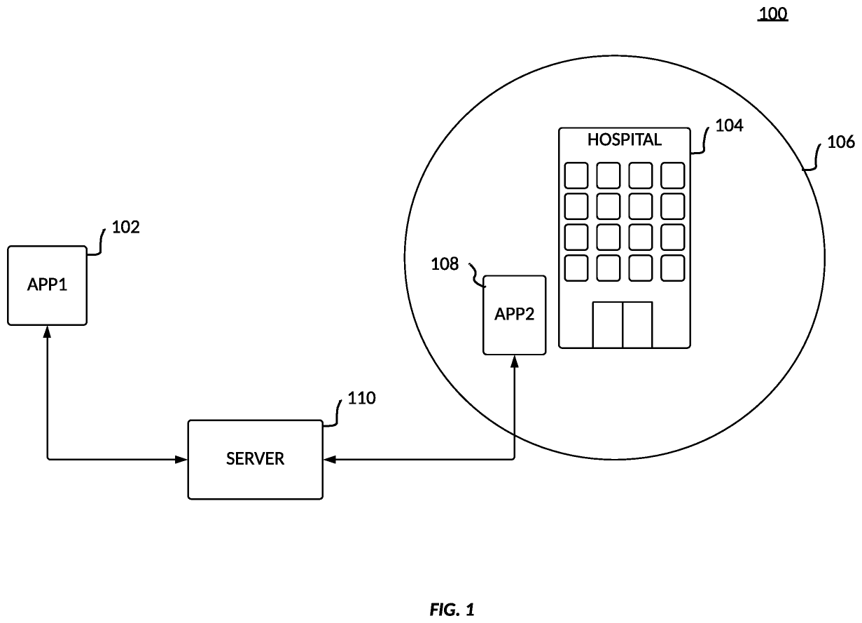

FIG. 1 is a block diagram of system 100 for geographically verifying patient logs in accordance with respective examples. A medical application, 102 or 108, may be used to collect patient logs. The medical application, 102 and 108, may run on a mobile device. The mobile device may have the ability to determine its location. For example, the mobile device may use the global positioning system, Wi-Fi positioning, dead reckoning, etc., to determine its location. The mobile device may make its location available to applications running on the mobile device. Accordingly, applications are able to determine and provide a location associated with the mobile application.

A medical student may use the medical application, 102 and 108, to log patient logs based on interactions with patients at a patient location, such as a hospital 104. Patient locations are locations where students may interact with patients. For example, medical students may interact with patients at medical centers, medical facilities, clinics, offices, etc., are patient locations. The medical application, 102 and 108, allows the medical student to check-in for a rotation at the hospital 104. The medical application 108 may send the check-in request to a server 110. The check-in request may include an identifier of the hospital 104. The server 110 may determine the location of the hospital 104 based on the hospital identifier. For example, the server 110 may have a stored list of hospital identifiers associated with the corresponding location of the hospital. As another example, the server 110 may query the location of the hospital 104 from an external data source. The server 110 may provide the hospital location to the medical application 102 and 108.

The medical application 102 and 108 allows a medical student to review their previous rotations and current rotations. Patient logs from previous rotations may be accessed, but new patient logs cannot be submitted. A medical student's active rotations may be accessed. The mobile application allows a medical student to enter new patient logs associated with the selected active rotation. In some embodiments, a medical student may access the features of the medical application via a web-based version of the medical application. Accordingly, a medical student may enter patient logs via the medical application on a mobile device or via a web browser.

In an example, the medical application 108 may determine its location as described above. The medical application 108 may then determine a distance between its location and the hospital location received from the server 110. If the medical application 108 is within a geographic range 106 of the hospital, the medical application 108 considers the patient log to be geographically verified. For example, if the medical application 108 is within two miles of the hospital, a submitted patient log is deemed to be geographically verified. In this context, geographically verified is determined based on the distance between the medical application 108 and the hospital 104. Another medical application 102 may be outside of the geographic range 106. Accordingly, submitted patient logs from the medical application 102 will include an indication that the patient log is not geographically verified.

In one example, the server 110 may provide the geographic range based on the hospital. For example, there may be two hospitals near one another such that the geographic range is smaller compared to other hospitals. In this example, the geographic range may be 1 mile, one-half of a mile, one-quarter of a mile, etc. In another example, a hospital may be spread out over a large campus such that the geographic range is 2.5 miles, 3 miles, 5 miles, etc.

The medical application 102 and 108 may be used to collect patient log information. Patient log information may include the student's initials, a patients gender, age, diagnosis, procedures, reason for visit, hospital, social history, family history, medical history, subjective, object, assessment, and planning (SOAP) notes, and one or more ICD-10 codes. Once collected, the patient log may be submitted to the server 110. In an example, prior to the patient log information being submitted to the server 110, the medical application 102 or 108 determines if the medical application 102 or 108 is within the hospital's geographic range 106. If the medical application 102 or 108 is within the geographic range 106, the patient log is submitted with a geographically verified indication. Otherwise, the patient log is submitted to the server 110 with an indication that patient log could not be geographically verified. In another example, the mobile application monitors its location and if at any time during the editing of a patient log the mobile application is within the geographic range 106, the geographically verified indication is set. This allows a geographically verified patient log to be edited while at the hospital 104 but submitted later outside of the geographic range 106.

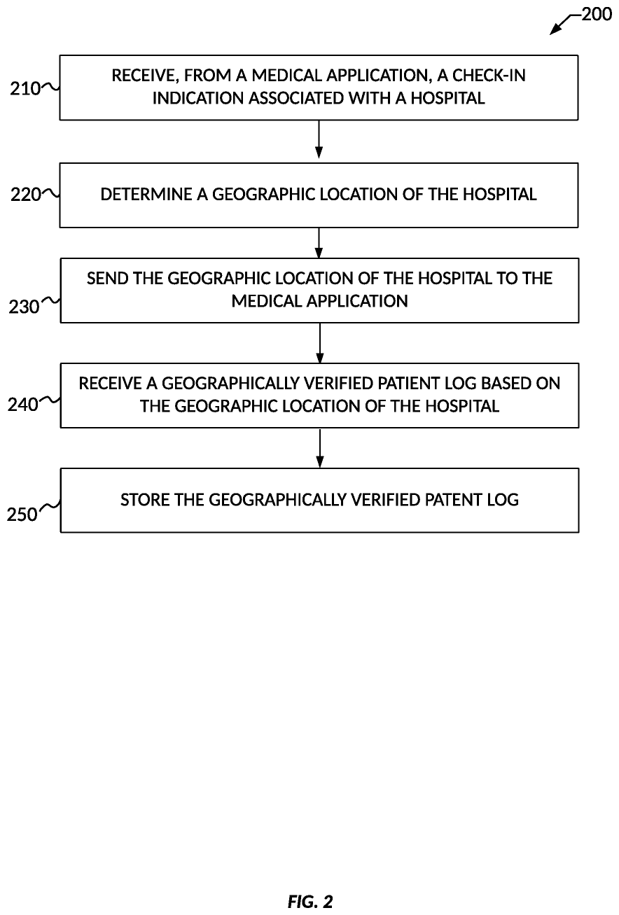

FIG. 2 is a flow diagram of process 200 for receiving geographically verified patient logs in accordance with respective examples. The process 200 may be executed on a computing device. At 210, a server receives a check-in indication from a medical application. The medical application indicates that a medical student, via a student identifier, is checking into a rotation at a particular hospital. The check-in indication include a hospital identifier that uniquely identifies the hospital. The check-in indication may also include a rotation identifier and a time stamp that indicates when the student checked into the hospital. The time stamp may also be determined by the server based on when the server receives the check-in indication.

At 220, a geographic location of the hospital is determined based on the hospital identifier. In an example, the hospital identifier is used to query a database that stores a hospital's geographic location associated with the hospital identifier. In another example, a database stores a hospital's address associated with the hospital identifier. The hospital's address may be used to determine the hospital's geographic location. For example, the hospital's address may be used as a query to a third-party service that returns the latitude and longitude of the address.

At 230, the server sends the medical application the geographic location of the hospital in response to the check-in indication. The hospital location may be sent to the medical application via a check-in confirmation response message.

The medical application may be used to collect patient log information. At 240, the server receives from the medical application, a geographically verified patient log. The patient log is geographically verified based on the geographic location of the hospital. For example, the medical application may determine its location and compare its location to the geographic location of the hospital. In an example, the medical application determines a distance between its location and the geographic location of the hospital. If the distance is less than a range, then the patient log is considered to be geographically verified. In this example, geographically verified means that the mobile application is near the hospital, i.e., within the range of the hospital.

In some examples, the medical application uses a set range such as 2 miles, 1 mile, ½ mile, etc. In other examples, the server may determine the geographic range for a check-in request. In these examples, the server sends the geographic range to the medical application. For example, different hospitals may have different ranges. In this example, the hospital identifier may be used to determine the geographic range for that hospital. In yet another example, the student identifier associated with the check-in request may be used to determine the range to use for verifying patient logs. In another example, a geographic range may be determined based on one or more of the hospital identifier, the rotation, the student identifier, the date, the time, or the day of the week. In some examples, multiple ranges may be found. For example, a range for the hospital and a second range associated with the student. In some examples, the smallest range is used and sent to the medical application.

The patient log may be geographically verified in different ways. In one example, the verification is done when the patient log is submitted. Thus, when a user of the medical application submits a patient log, the medical application determines if the medical application is within the geographic range of the hospital. In another example, the medical application monitors its location when the user is entering information into a patient log. If at any point during the data entry the user is within the hospital's range, the patient log is considered geographically verified. This feature is useful for students who enter some data while within range of the hospital and then later submits the patient log when outside of the range.

The received patient log includes an indication regarding if the patient log is geographically verified. For example, a Boolean flag may be used to represent the geographically verified status. As another example, the patient log may include the calculated distance. The distance calculation may be used by the server to determine the geographically verified status. In yet another example, the patient log includes both a geographically verified indication and the distance value.

At 250, the server stores the geographically verified patient log. The server may store multiple patient logs associated with various students, rotations, hospitals, programs, etc. The patient logs may be retrieved by supervising doctors, clerkship administrators, or the students themselves. For example, the server may receive a request for patient logs associated with a specific rotation at a hospital. The request may originate via a web portal. For example, a supervising doctor or clerkship administrator may log into the web portal and make the request for patient logs. The patient logs associated with that rotation may be searched for, retrieved, and provided. The patient logs include the geographically verified indication.

The stored patient logs may also be used to determine students that are submitting patient logs onsite versus offsite. For example, geographically unverified patient logs may be determined. Geographically verified patient logs may also be determined. The patient logs may be specific to a hospital, a rotation, etc. The retrieved patient logs may be used to determine, for each student, the percentage of patient logs that were geographically verified. If that percentage is below a threshold, such as 90% 80%, 75%, 50%, etc., the student may be flagged. The flagged students may receive automatic emails, text messages, etc., reminding them to submit their patient logs at the respective hospital.

The server may also receive a check-out indication at the end of a student's shift. A time stamp associated with the received check-out indication may be stored. A shift duration for a rotation may be determined using the time stamp associated with the check-in indication and the time stamp associated with the check-out indication. The duration may be stored for later use.

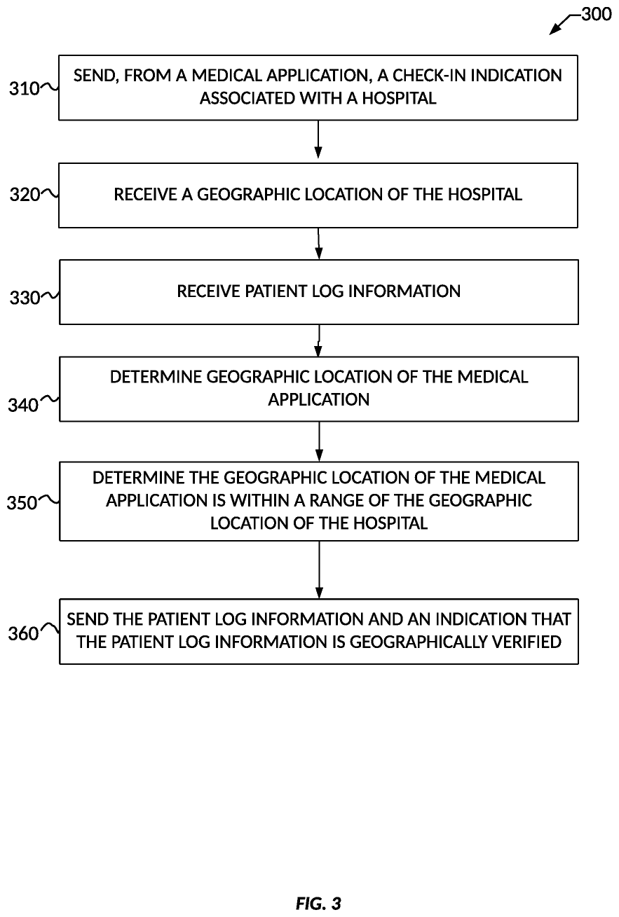

FIG. 3 is a flow diagram of a process 300 at a medical application for providing geographically verified patient logs in accordance with respective examples. The process 300 may be executed on a computing device. At 310, the medical application sends to a medical server, a check-in indication associated with a student. The check-in indication includes a hospital identifier that identifies a hospital. The check-in indication may also include a student identifier and a rotation identifier. A time stamp may be included with the check-in indication as well. At 320, in response to the check-in indication, the medical application receives a geographic location of the hospital in response from the medical server.

The medical application may be used to collect patient log information from a student. For example, a student may enter information regarding an interaction with a patient. At 330, the medical application receives patient log information. Next, the medical application determines if the patient log information can be geographically verified. At 340, a geographic location of the medical application is determined. The geographic location may be determined from GPS or any other location service provider available to the medical application. The medical application may then determine a distance between the hospital location and the location of the medical application. At 350, the medical application determines if the medical application is within a range of the geographic location of the hospital. The determined distance between the medical application and the hospital may be used to determine if the patient log information is considered geographically verified. At 360, the medical application sends the patient log information and an indication that the patient log information is geographically verified to the medical server.

The medical application may receive a request for a patient log entry form. The patient log entry form allows a student to enter the patient log information. The patient log entry form may have a number of international classification of diseases (ICD) codes, such as ICD-10 codes, embedded within the patient log entry form. In one example, a text listing of ICD codes are converted into a resource that is part of the medical application. The resource may then be accessed by the medical application as an assembly that is within the medical application. This allows the medical application to quickly access thousands of ICD codes without having to request the codes from an external source. After received the request for the patient log entry form, the medical application displays the patient log entry form within the medical application. The student may then interact with the patient log entry form to entry data related to a patient encounter. Upon entering all of the relevant patient log information, the student may submit the patient log information to the medical server for storage.

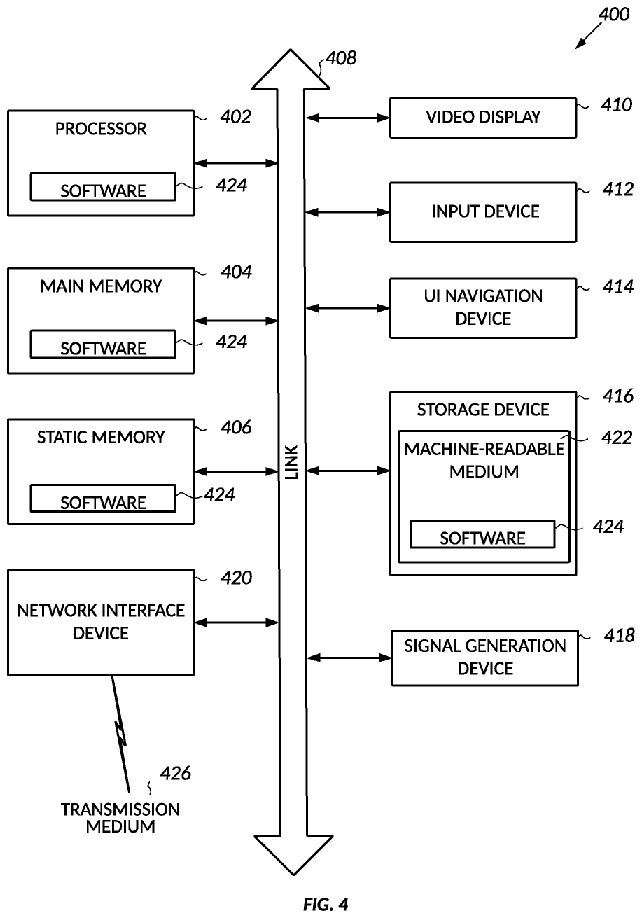

FIG. 4 is an example computing device that can be used in conjunction with the technologies described herein. In alternative embodiments, the computing device 400 may operate as a standalone device or may be connected (e.g., networked) to other computing devices. In a networked deployment, the computing device 400 may operate in the capacity of a server communication device, a client communication device, or both in server-client network environments. The computing device 400 may be a personal computer (PC), a tablet PC, a set top box (STB), a personal digital assistant (PDA), a mobile telephone, a smart phone, a web appliance, a network router, switch or bridge, or any computing device capable of executing instructions (sequential or otherwise) that specify actions to be taken by that computing device. Further, while only a single computing device is illustrated, the term “computing device” shall also be taken to include any collection of computing devices that individually or jointly execute a set (or multiple sets) of instructions to perform any one or more of the methodologies discussed herein, such as cloud computing, software as a service (SaaS), other computer cluster configurations. Computing device may be an implementation of device 102 and 110 and may perform the process of FIG. 2 and FIG. 3.

Computing device 400 may include a hardware processor 402 (e.g., a central processing unit (CPU), a graphics processing unit (GPU), a hardware processor core, or any combination thereof), a main memory 404 and a static memory 406, some or all of which may communicate with each other via a link (e.g., bus) 408. The computing device 400 may further include a display unit 410, an input device 412 (e.g., a keyboard), and a user interface (UI) navigation device 414 (e.g., a mouse). In an example, the display unit 410, input device 412, and UI navigation device 414 may be a touch screen display. In an example, the input device 412 may include a touchscreen, a microphone, a camera (e.g., a panoramic or high-resolution camera), physical keyboard, trackball, or other input devices.

The computing device 400 may additionally include a storage device 416, a signal generation device 418 (e.g., a speaker, a projection device, or any other type of information output device), a network interface device 420, and one or more sensors, such as a global positioning system (GPS) sensor, compass, accelerometer, motion detector, or other sensor. The computing device 400 may include an input/output controller 428, such as a serial (e.g., universal serial bus (USB), parallel, or other wired or wireless (e.g., infrared (IR), near field communication (NFC), etc.) connection) to communicate or control one or more peripheral devices (e.g., a printer, card reader, etc.) via one or more input/output ports.

The storage device 416 may include a computing-readable (or machine-readable) storage media 422, on which is stored one or more sets of data structures or instructions 424 (e.g., software) embodying or utilized by any one or more of the techniques or functions described herein. In an example, at least a portion of the software may include an operating system and/or one or more applications implementing one or more of the functionalities described herein. The instructions 424 may also reside, completely or at least partially, within the main memory 404, within the static memory 406, and/or within the hardware processor 402 during execution thereof by the computing device 400. In an example, one or any combination of the hardware processor 402, the main memory 404, the static memory 406, or the storage device 416 may constitute computing device (or machine) readable media.

While the computer-readable storage media 422 is illustrated as a single medium, a “computer-readable storage media” or “machine-readable storage media” may include a single medium or multiple media (e.g., a centralized or distributed database, and/or associated caches and servers) configured to store the one or more instructions 424.

In an example, a computer-readable storage media or machine-readable storage media may include any medium that is capable of storing, encoding, or carrying instructions for execution by the computing device 400 and that cause the computing device 400 to perform any one or more of the techniques of the present disclosure, or that is capable of storing, encoding or carrying data structures used by or associated with such instructions. Non-limiting computer-readable storage media examples may include solid-state memories, and optical and magnetic media. Specific examples of computer-readable storage media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and optical media disks. The computer-readable storage media is non-transitory in that the storage media does not consist of transitory propagating signals.

The instructions 424 may further be transmitted or received over a communications network 426 using a transmission medium via the network interface device 420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. The network interface device 420 may use the transfer protocols to transmit data using transitory propagating signals.

In an example, the network interface device 420 may include one or more physical jacks (e.g., Ethernet, coaxial, or phone jacks) or one or more antennas to connect to the communications network 426. In an example, the network interface device 420 may include one or more wireless modems, such as a Bluetooth modem, a Wi-Fi modem or one or more modems or transceivers operating under any of the communication standards mentioned herein. In an example, the network interface device 420 may include a plurality of antennas to wirelessly communicate using at least one of single-input multiple-output (SIMO), multiple-input multiple-output (MIMO), or multiple-input single-output (MISO) techniques. In some examples, the network interface device 420 may wirelessly communicate using Multiple User MIMO techniques. In an example, a transmission medium may include any intangible medium that is capable of storing, encoding or carrying instructions for execution by the computing device 400, and includes digital or analog communications signals or like communication media to facilitate communication of such software.

Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

The above description is intended to be illustrative, and not restrictive. For example, the above-described examples (or one or more aspects thereof) may be used in combination with others. Other embodiments may be used, such as by one of ordinary skill in the art upon reviewing the above description. Further, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

What is claimed is:

1. A method for receiving geographically verified patient logs, the method comprising operations performed using an electronic processor, the operations comprising:
receiving, from a medical application, a check-in indication associated with a student and a hospital, wherein the check-in indication comprises a hospital identifier that identifies the hospital;
determining, based on the hospital identifier, a geographic location of the hospital;
sending, to the medical application, the geographic location of the hospital in response to the check-in indication;
receiving, from the medical application, a geographically verified patient log, wherein the patient log is geographically verified based on the geographic location of the hospital, and wherein the geographically verified patient log comprises a geographically verified indication; and
storing the geographically verified patient log.

2. The method of claim 1, wherein the geographically verified patient log is verified when the medical application is within two miles of the geographic location of the hospital when the patient log is submitted.

3. The method of claim 1, further comprising:
determining a geographic range for geographical verification based on the hospital identifier; and
sending, to the medical application, the geographic range, wherein the patient log is geographically verified when the medical application is within the geographic range of the hospital when the patient log is submitted.

4. The method of claim 1, wherein the geographically verified patient log is verified when the medical application is within two miles of the geographic location of the hospital at any time when the geographically verified patient log is edited or submitted.

5. The method of claim 1, further comprising:
receiving a request for patient logs associated with a rotation at the hospital; and
providing the patient logs, wherein the patient logs comprise the geographically verified patient log.

6. The method of claim 1, further comprising:
searching stored patient logs for geographically unverified patient logs;
searching stored patient logs for geographically verified patient logs;
determining a percentage of geographically verified patient logs for each of a plurality of students; and
identifying students whose percentage of geographically verified patients logs are below a threshold value.

7. The method of claim 1, further comprising:
storing a first timestamp associated with receipt of the check-in indication;
receiving a checkout-out indication;
storing a second timestamp associated with receipt of the check-out indication; and
determining a shift duration based on the first time stamp and the second timestamp.

8. A non-transitory computer-readable storage medium storing computer-executable instructions for receiving geographically verified patient logs, the stored instructions comprising:
instructions to receive, from a medical application, a check-in indication associated with a student, wherein the check-in indication comprises a hospital identifier that identifies a hospital;
instructions to determine, based on the hospital identifier, a geographic location of the hospital;
instructions to send, to the medical application, the geographic location of the hospital in response to the check-in indication;
instructions to receive, from the medical application, a geographically verified patient log, wherein the patient log is geographically verified based on the geographic location of the hospital; and
instructions to store the geographically verified patient log.

9. The non-transitory computer-readable storage medium of claim 8, wherein the geographically verified patient log is verified when the medical application is within two miles of the geographic location of the hospital when the patient log is submitted.

10. The non-transitory computer-readable storage medium of claim 8, wherein the instructions further comprise;
instructions to determine a geographic range for geographical verification based on the hospital identifier; and
instructions to send, to the medical application, the geographic range, wherein the patient log is geographically verified when the medical application is within the geographic range of the hospital when the patient log is submitted.

11. The non-transitory computer-readable storage medium of claim 8, wherein the geographically verified patient log is verified when the medical application is within two miles of the geographic location of the hospital at any time when the geographically verified patient log is edited or submitted.

12. The non-transitory computer-readable storage medium of claim 8, wherein the instructions further comprise:
instructions to receive a request for patient logs associated with a rotation at the hospital; and
instructions to provide the patient logs, wherein the patient logs comprise the geographically verified patient log.

13. The non-transitory computer-readable storage medium of claim 8, wherein the instructions further comprise:
instructions to search stored patient logs for geographically unverified patient logs;
instructions to search stored patient logs for geographically verified patient logs;
instructions to determine a percentage of geographically verified patient logs for each of a plurality of students; and
instructions to identify students whose percentage of geographically verified patients logs are below a threshold value.

14. The non-transitory computer-readable storage medium of claim 8, wherein the instructions further comprise:
instructions to store a first time stamp associated with receipt of the check-in indication;
instructions to receive a checkout-out indication;
instructions to store a second timestamp associated with receipt of the check-out indication; and
instructions to determine a shift duration based on the first timestamp and the second timestamp.

15. A system for providing geographically verified patient logs, the system comprising:
one or more electronic processors configured to:
send, to a medical server from a medical application, a check-in indication associated with a student, wherein the check-in indication comprises a hospital identifier that identifies a hospital;
receive, from the medical server, a geographic location of the hospital in response to the check-in indication;
receive patient log information;
determine a geographic location of the medical application;
determine the geographic location of the medical application is within a geographic range of the geographic location of the hospital; and
send, to the medical server, the patient log information and an indication that the patient log information is geographically verified based on the determination that the medical application is within the geographic range of the geographic location of the hospital.

16. The system of claim 15, further comprising:
receive, from the medical application, a request for a patient log entry form;
embed a plurality of international classification of diseases (ICD) codes in the patient log entry form, wherein the ICD codes are referenced from an assembly within the medical application; and
display the patient log entry form within the medical application.

17. The system of claim 15, wherein the geographically verified patient log is verified when the medical application is within two miles of the geographic location of the hospital when the patient log is submitted.

18. The system of claim 15, wherein the one or more electronic processors are further configured to receive, from the medical server, the geographic range, wherein the geographic range is based on the hospital range.

19. The system of claim 15, wherein the geographically verified patient log is verified when the medical application is within two miles of the geographic location of the hospital at any time when the geographically verified patient log is edited or submitted.

20. The system of claim 15, wherein the one or more electronic processors are further configured to:
send a request for patient logs associated with a rotation at the hospital; and
receive the patient logs, wherein the patient logs comprise the geographically verified patient log.
